SEQUENCE LISTING (36 sequences)

SEQ ID NO: 1 - DNA, 20 bp, Artificial Sequence
  Primer
atgactctacccacggcaag

SEQ ID NO: 2 - DNA, 20 bp, Artificial Sequence
  primer
ggaagatggtgatgggtttc

SEQ ID NO: 3 - DNA, 21 bp, Artificial Sequence
  primer
actttatcttcggtcagagtg

SEQ ID NO: 4 - DNA, 20 bp, Artificial Sequence
  primer
ctcacgacatccaggactga

SEQ ID NO: 5 - DNA, 21 bp, Artificial Sequence
  primer
gagagagattcgcactcagta

SEQ ID NO: 6 - DNA, 20 bp, Artificial Sequence
  primer
tgaggtctgcaaacttggac

SEQ ID NO: 7 - DNA, 20 bp, Artificial Sequence
  primer
gaaggtctccagcgagtgag

SEQ ID NO: 8 - DNA, 20 bp, Artificial Sequence
  primer
tcaagcagcacagaagagga

SEQ ID NO: 9 - DNA, 21 bp, Artificial Sequence
  primer
cacaagaactacccactacgg

SEQ ID NO: 10 - DNA, 20 bp, Artificial Sequence
  primer
gggtgtacgaggtgtcacaa

SEQ ID NO: 11 - DNA, 20 bp, Artificial Sequence
  primer
aggggcttatctctcaccgt

SEQ ID NO: 12 - DNA, 20 bp, Artificial Sequence
  primer
aaccagagatgtggcttccg

SEQ ID NO: 13 - DNA, 20 bp, Artificial Sequence
  primer
tggaaatggacggacaaggg

SEQ ID NO: 14 - DNA, 20 bp, Artificial Sequence
  primer
tgggaatctcaccaatgccc

SEQ ID NO: 15 - DNA, 20 bp, Artificial Sequence
  primer
gttaaccacagcaaggcagc

SEQ ID NO: 16 - DNA, 20 bp, Artificial Sequence
  primer
tcggctactgtcaacaaccc

SEQ ID NO: 17 - DNA, 20 bp, Artificial Sequence
  primer
gaaccccgaaaggtgtggat

SEQ ID NO: 18 - DNA, 20 bp, Artificial Sequence
  primer
ttcgatttgaggtgctcgct

SEQ ID NO: 19 - DNA, 20 bp, Artificial Sequence
  primer
gccacactagtttccctgct

SEQ ID NO: 20 - DNA, 20 bp, Artificial Sequence
  primer
atcagaacttggtgcctcgg

SEQ ID NO: 21 - DNA, 20 bp, Artificial Sequence
  primer
cacacaagtggtccacgaga

SEQ ID NO: 22 - DNA, 20 bp, Artificial Sequence
  primer
gctccgagaaggtgtactgg

SEQ ID NO: 23 - DNA, 20 bp, Artificial Sequence
  primer
tgtgtggagcctttctctgc

SEQ ID NO: 24 - DNA, 20 bp, Artificial Sequence
  primer
cccaggagatatacggcacg

SEQ ID NO: 25 - DNA, 20 bp, Artificial Sequence
  primer
tacttcatcgtccacgcagc

SEQ ID NO: 26 - DNA, 20 bp, Artificial Sequence
  primer
acaatggtgtacggtggcat

SEQ ID NO: 27 - DNA, 20 bp, Artificial Sequence
  primer
gtccacacctctgtctacgc

SEQ ID NO: 28 - DNA, 20 bp, Artificial Sequence
  primer
ggacttgagcgagccttgaa

SEQ ID NO: 29 - DNA, 20 bp, Artificial Sequence
  primer
caatgcctccaaattgtcct

SEQ ID NO: 30 - DNA, 20 bp, Artificial Sequence
  primer
tccatcctcatccgttcttc

SEQ ID NO: 31 - DNA, 20 bp, Artificial Sequence
  primer
gccacggataccatgaacga

SEQ ID NO: 32 - DNA, 20 bp, Artificial Sequence
  primer
agctgacattgtgccactga

SEQ ID NO: 33 - DNA, 20 bp, Artificial Sequence
  primer
gggctcttgtgagcatctgt

SEQ ID NO: 34 - DNA, 20 bp, Artificial Sequence
  primer
tgttcagggtcccatttcgg

SEQ ID NO: 35 - DNA, 20 bp, Artificial Sequence
  primer
ggtggggaacggactcattt

SEQ ID NO: 36 - DNA, 20 bp, Artificial Sequence
  primer
ccaaaggaccacacgtcaga

(19) United States
(12) Patent Application Publication
(10) Pub. No.: US 2017/0130194 A1
(43) Pub. Date: May 11, 2017

(54) GUIDING STEM CELL DIFFERENTIATION USING GRAPHENE-NANOFIBER HYBRID SCAFFOLDS

(71) Applicant: Rutgers, The State University of New Jersey, New Brunswick, NJ (US)

(72) Inventors: Ki-Bum Lee, Monmouth Junction, NJ (US); Shreyas Shah, Dayton, NJ (US)

(73) Assignee: Rutgers, The State University of New Jersey, New Brunswick, NJ (US)

(21) Appl. No.: 15/303,151

(22) PCT Filed: Apr. 10, 2015
(86) PCT No.: PCT/US2015/025330

§ 371 Date: Oct. 10, 2016

Related U.S. Application Data
(60) Provisional application No. 61/978,177, filed on Apr. 10, 2014.

(51) Int. Cl. C12N 5/00; C12N 5/074; C12N 5/0775; D06M 11/76; A61L 27/18; A61L 27/38; A61K 35/545; C12N 5/0797; A61L 27/30

(52) U.S. Cl. CPC C12N 5/0068; C12N 5/0623; C12N 5/0696; C12N 5/0667; A61L 27/306; A61L 27/18; A61L 27/3834; A61K 35/545; D06M 11/76; C12N 2533/30; A61L 2400/12; D06M 2101/32

(57) ABSTRACT

The present invention relates to graphene oxide-coated nanofiber scaffold that provides instructive physical cues to the differentiation of stem cells into selected cell lineages or networks.

### CROSS-REFERENCE TO RELATED APPLICATIONS

This application claims priority under 35 USC §119(e) to U.S. Provisional Patent Application Ser. No. 61/978,177, filed on Apr. 10, 2014, which is hereby incorporated by reference in its entirety.

The present invention was made with government support under grant numbers 1DP20D006462-01 and 1R21NS085569-01, both awarded by the National Institute of Health, and 09-3085-SCR-E-0, awarded by the State of New Jersey. The United States government and the State of New Jersey have certain rights in this invention.

### FIELD OF THE INVENTION

The present invention is directed to scaffolds and devices for tissue engineering and methods having micro/nano-structures in combination with graphene-based nanomaterials for the selective differentiation of various types of stem cells. More specifically, the present invention is directed to a platform which uses specific combinations of the graphene-based nanomaterials with various sized micro/nano-structure substrates made by various methods that provide instructive physical cues for a defined type of cell desired via differentiation.

### BACKGROUND

Stem cell therapy offers a promising new option for the treatment of human disease. Adult stem cells have been used successfully to treat patients in various clinical trials across a number of clinical conditions. For example, mesenchymal stem cells (MSCs) have been used to treat a number of conditions in animal models and are currently being evaluated in clinical trials to treat various diseases. Damage to the central nervous system (CNS) from degenerative diseases or traumatic injuries is particularly devastating due the limited regenerative capabilities of the CNS. Among the current approaches, stem cell-based regenerative medicine has shown great promise in achieving significant functional recovery by taking advantage of the self-renewal and differentiation capabilities of stem cells, which include pluripotent stem cells (PSCs), mesenchymal stem cells (MSCs) and neural stem cells (NSCs). Meanwhile, numerous types of natural and synthetic biomaterial scaffolds have been developed, the two main classes being hydrogels and nanofibers, in an attempt to mimic the cellular microenvironment, support cellular growth and improve cellular viability.

However, many of the materials require the administration of multiple growth factors to promote stem cell differentiation, and bioactive scaffolds or implants still suffer from severe limitations including potential pathogenic infections, low availability and high costs. In addition, many modern approaches also face further challenges when it comes to scalability and compatibility with implants.

Therefore, there remains a significant need for the development of novel materials and scaffolds that allow for selective stem cell differentiation and better biocompatibility.

### SUMMARY

The present invention relates to scaffolds and devices for tissue engineering and methods having micro/nano-structures in combination with graphene-based nanomaterials for the selective differentiation of various types of stem cells. The scaffolds and devices can be applied to the treatment of various injuries or disorders by promoting tissue-specific stem cell differentiation.

An aspect of the invention provides scaffolds for tissue engineering comprising nanofibers coated with graphene oxide, wherein said nanofibers have an average diameter in the range of about 100 nm-3 μm.

In some embodiments, the nanofibers comprise a polymer selected from the group consisting of polyglycolic acid (PGA), polylactic acid (PLA), poly(lactic-co-glycolic acid) (PLGA), poly-ε-caprolactone (PCL), polyanhydride, polyorthoesters, polyvinylalcohol, polyethylene glycol, polyurethane, polyacrylic acid, poly-N-isopropyl acrylamide, poly(ethylene oxide)-poly(propylene oxide)-poly(ethylene oxide)copolymers, derivatives thereof, and copolymers thereof. In some embodiments, the polymer is PCL.

In some embodiments, the lateral dimension of the graphene oxide is in the range of about 50-1000 nm.

In some embodiments, the graphene oxide is saturated on the nanofibers.

In some embodiments, the scaffold further includes stem cells seeded in the scaffold. In some embodiments, the stem cells are selected from the group consisting of pluripotent stem cells (PSCs), mesenchymal stem cells (MSCs) and neural stem cells (NSCs).

In some embodiments, the scaffold further includes differentiated stem cells seeded in the scaffold. In some embodiments, the differentiated stem cells are oligodendrocytes.

Another aspect of the present invention provides an implantable medical device containing the scaffold of the present invention.

Another aspect of the invention provides a method of directing stem cell differentiation comprising exposing the scaffold of the present invention to a culture media comprising stem cells for a period of time sufficient to allow the stem cells to differentiate into cells of interest.

In some embodiments, the stem cells of the above method are selected from the group consisting of pluripotent stem cells (PSCs), mesenchymal stem cells (MSCs) and neural stem cells (NSCs).

In some embodiments, the stem cells differentiate into chondrocytes, osteoblasts, neurons, oligodendrocytes, astocytes, and microglial cells.

In some embodiments, the culture media does not contain growth factors or external stimulation. In some embodiments, the culture media contains growth factors such as epidermal growth factor (EGF) and basic fibroblast growth factor (bFGF).

Another aspect of the present invention provides a method of treating an injury or disorder comprising implanting the scaffold of the present invention in a subject in need, wherein the scaffold is seeded with stem cells or differentiated stem cells. In some embodiments, the subject is human.

In some embodiments, the stem cells are selected from the group consisting of pluripotent stem cells (PSCs), mesenchymal stem cells (MSCs) and neural stem cells (NSCs).

In some embodiments, the differentiated stem cells comprise at least one member selected from the group consisting of osteocytes, chondrocytes, osteoblasts, fibroblasts, keratinocytes, adipocytes, tenocytes, myocytes, hepatocytes, neurons, oligodendrocytes, astocytes, microglial cells, muscles cells, nerve cells and cardiac myocytes.

Another aspect of the present invention provides a method of preparing a scaffold of for tissue engineering, comprising contacting nanofibers with a solution of graphene oxide, wherein the concentration of the graphene oxide is optionally adjusted to control the thickness of the coating. In some embodiments, the graphene oxide deposited on the nanofibers reaches saturation.

Another aspect of the invention provides a method of dedifferentiating lineage committed mammalian cells into induced pluripotent stem cells (iPS cells), comprising seeding lineage committed mammalian cells in the scaffold of the present invention and exposing the scaffold to a culture medium for a sufficient period of time to allow dedifferentiation of the cells. In some embodiments, the lineage committed mammalian cells comprise at least one member selected from the group consisting of osteocytes, chondrocytes, osteoblasts, fibroblasts, keratinocytes, adipocytes, tenocytes, myocytes, hepatocytes, neurons, oligodendrocytes, astocytes, microglial cells, muscles cells, nerve cells and cardiac myocytes. Also provided is a iPS cell produced according to the above method. A method of treating a disease or disorder using the iPS cells produced according to the method of present invention is also described.

### BRIEF DESCRIPTION OF THE DRAWINGS

FIG. 1 illustrates the fabrication and application of graphene oxide-nanofiber hybrid scaffolds in directing stem cell differentiation.

### DETAILED DESCRIPTION

Various embodiments provide scaffolds for tissue engineering that contain graphene oxide-coated nanomaterials that provide instructive physical cues to the differentiation of stem cells into selected cell lineages or networks. In particular, graphene oxide of the scaffolds demonstrates synergistic effect in promoting stem cell differentiation when used in combination with materials such as nanofibers. An additional benefit of graphene oxide is its ability to affect the absorption of biomolecules, which is otherwise difficult to achieve using other types of ECM and/or carbon-based nanomaterials. The scaffolds can be applied to the treatment of various injuries or disorders by promoting tissue-specific stem cell differentiation.

Throughout this patent document, various publications are referenced. The disclosures of these publications in their entireties are hereby incorporated by reference into this application in order to more fully describe the state of the art to which the disclosed matter pertains. While the following text may reference or exemplify specific elements of a composite or a method of utilizing the composite, it is not intended to limit the scope of the invention to such particular reference or examples. Various modifications may be made by those skilled in the art, in view of practical and economic considerations, such as the size and composition of the nanofiber and culturing conditions for differentiating the stem cells.

The articles “a” and “an” as used herein refers to “one or more” or “at least one,” unless otherwise indicated. That is, reference to any element or component of the present invention by the indefinite article “a” or “an” does not exclude the possibility that more than one of the element or component is present.

The term “about” as used herein refers to the referenced numeric indication plus or minus 10% of that referenced numeric indication.

The term “lineage committed cell” as used herein refers to any cell that has or will differentiate into a particular cell type or related cell types. Non-limiting examples of lineage committed cells include osteocytes, chondrocytes, fibroblasts, keratinocytes, adipocytes, tenocytes, myocytes, hepatocytes, neurons, oligodendrocytes, astocytes, and microglial cells.

The term “differentiate” or “differentiation” as used herein refers to the process by which precursor or progenitor cells differentiate into specific cell types such as osteocytes and chondrocytes.

The term “dedifferentiate” or “dedifferentiation” as used herein refers to the process by which lineage committed cells (e.g. myoblasts or osteoblasts) reverse their lineage commitment and become precursor or progenitor cells (i.e. multipotent or pluripotent stem cells).

The term “induced pluripotent stem (iPS) cells” as used herein refers to cells having properties similar to other pluripotent stem cells, e.g., hES cells, hEG cells, pPS (primate pluripotent stem) cells, parthenogenic cells and the like.

### Scaffolds and Devices

An aspect of the invention provides a scaffold for tissue engineering containing a nanomaterial coated with graphene oxide. The nanomaterial has an average size in the range of about 100 nm-3 μm.

Either or both of graphene oxide and the substrate can be seeded with stem cells or differentiated stem cells. In some embodiments, the stem cells are grown on the scaffold in an appropriate culture medium under conditions that do not require implementation with growth factors or external stimulation, or combinations thereof. In some embodiments, the culture medium include growth factors or external stimulation. In some embodiments the stem cells on the scaffold are grown and differentiated in vitro. In some embodiments, the scaffold can be incorporated into a device for implantation to treat an injury or disorder in a subject.

Suitable nanomaterials may contain gold, metal, metal oxide, polymer, titanium dioxide, silver, carbon nanotubes, hydroxyapatite, quantum dots, crystals, salts, ceramic materials, magnetic materials, or any combination thereof. In some embodiments, the nanomaterial contains a polymer which can be artificial or synthetic. The size or diameter of the nanomaterial ranges from 100 nm-3 μm, all subunits included. Non-limiting examples of size or diameters include about 100, 200, 300, 400, 500, 600, 700, 800, 900, 1000, 1200, 1400, 1600, 1800, 2000, 2200, 2400, 2600, 2800 and 3000 nm.

In some embodiments, the nanomaterial is a nanofiber. Nanofibers may attain a high surface area comparable with the coating with graphene oxide and other suitable materials, yet are fairly flexible, and retain one macroscopic dimension which makes them easy to handle, orient and organize. Moreover, the high surface area of nanofibers may facilitate the addition of particles that improve the properties of the nanofibers such as mechanical strength, and/or impart additional functionality such as therapeutic activity, catalytic activity, or micro-electronic/optoelectronic functionality. Further, the 3-dimensional structure of the graphene oxide-coated nanofiber scaffold of the present invention provide physical cues to the growth of certain tissues such as axons due to the close morphological resemblance.

In some embodiments, the nanofiber is fabricated from a polymer. Preferably, the nanofiber contains a biocompatible polymer such as polyimide, polyamide, and polycarbonate, which are suitable for in vitro and in vivo applications. Non-limiting examples of the suitable polymer include polyglycolic acid (PGA), polylactic acid (PLA), poly(lactic-co-glycolic acid) (PLGA), poly-ε-caprolactone (PCL), polydimethylsiloxane (PDMS), polyanhydride, poly-orthoesters, polyvinylalcohol, polyethylene glycol, polyurethane, polyacrylic acid, poly-N-iso-propyl acrylamide, poly(ethylene oxide)-poly(propylene oxide)-poly(ethylene oxide)copolymers, derivatives thereof, and copolymers thereof. In some embodiments, the nanofiber forms a structure or scaffold that is three-dimensional.

In some embodiments, the nanofiber may optionally be used in combination with another material suitable for the differentiation of stem cells. For example, hydrogels are known to promote stem cell differentiation. Integration of a hydrogel into the scaffold of the present invention may provide additional benefits such as enhancing conversion rate and selectivity of stem cell differentiation. Hydrogels may be formed of any components within the purview of those skilled in the art. Hydrogels including various types of collagen and denatured collagen are available from natural sources or artificial means, including for example polypeptide-based hydrogels, polysaccharide-based hydrogels, and petrochemical-based hydrogels.

The diameter of the nanofibers can be adjusted to suit the need of the selective differentiation of the stem cells. For example, an average diameter of 200-300 nm is a fiber size range that has been reported to be favorable for oligodendrocyte culture, potentially due to the close morphological resemblance to axons. The ranges of the nanofiber diameter include for example about 50-100, 100-150, 150-200, 200-250, 250-300, 300-350, 350-400, 400-450, 450-500, 500-700, 700-1000, 1000-1500, 1500-2000, 2000-2500 and 2500-3000 nm. Non-limiting examples of the diameter of the nanofiber include about 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 600, 700, 800, 900, 1000, 1200, 1400, 1600, 1800, 2000, 2200, 2400, 2600, 2800 and 3000 nm.

Various high volume and low cost methods for the production of nanofibers are known in the art, including for example, drawing, phase separation, electrospinning, template synthesis and self-assembly. Of these, melt blowing, splitting/dissolving of bicomponent fibers, and electrospinning have shown a potential for commercial-scale fiber production. The first two techniques are based on mechanical drawing of melts and are well-established in high-volume manufacturing. In melt blowing polymers are extruded from dies and stretched to smaller diameters by heated, high velocity air streams. Bicomponent spinning involves extrusion of two immiscible polymers and two-step processing: (1) melt spinning the two polymer melts through a die with a “segmented pie” or “islands-in-the-sea” configuration, followed by solidification and (2) release of small filaments by mechanically breaking the fiber or by dissolving one of the components. Various modifications of the fabrication of nanofibers have also been reported including for example US Pat. App. 20130012598 and PCT/US2014/051267, the entire disclosure of which is hereby incorporated by reference.

In some embodiments, electrospinning technique is used in the production of nanofibers of the present invention. Electrospinning differs from melt or dry spinning by the physical origin of the electrostatic rather than mechanical forces being used to draw the fibers.

Nanofibers can be coated in various known approaches. In an exemplary embodiment, the graphene oxide is dispersed in deionized water at suitable concentrations. The nanofibers (e.g. PCL nanofibers) can be treated with oxygen plasma to increase its affinity by introducing hydrophilic groups. Subsequent deposition of the graphene oxide solution directly on top of the nanofibers and vacuum drying afford a desired coating on the nanofibers. The concentration of the graphene oxide solution can be adjusted so that the thickness or extent of the coating on the substrate is controllable. In some embodiments, the coating of graphene oxide on the nanofiber reaches a saturation point.

Graphene oxide of the present invention can be readily prepared by various methods such as a modified Hummer's method (Xu, et al. J. Am. Chem. Soc. 2008, 130, 5856-5857). The size of the graphene oxide can be further controlled by filtration. In some embodiments, the lateral dimension of the graphene oxide of the present invention ranges from about 10-100, 100-150, 150-200, 200-250, 250-300, 300-350, 350-400, 400-500, 500-600, 600-700, 700-800, 800-900 and 900-1000 nm, all sub-ranges include. Exemplary embodiments of the average size of the graphene oxide include about 50, 80, 100, 120, 140, 160, 180, 200, 220, 240, 260, 280, 300, 320, 340, 360, 380, 400, 500, 600, 700, 800, 900 and 1000 nm.

The highly oxidized nature of the graphene oxide contributes significantly to the attachment of the graphene oxide onto the nanofibers. Non-limiting examples of the ratio between carbon and oxygen include about 10:8, 10:7, 10:6, 10:5, 6:4, 7:3, and 8:2. In some embodiments, the graphene oxide is attached to the substrate without the use of any chemical linkers and/or additional processes.

Scaffolds for tissue engineering containing graphene oxide attached to the substrate described herein exhibit long-term stability which is highly important as it prevents possible adverse effects such as the release of graphene oxide from the surface while stem cells are being differentiated. In addition, this long-term stability facilitates the stable interaction of the extracellular matrix (ECM) environment with stem cells over extended periods of time, which is required during differentiation. In some embodiments, graphene oxide can be attached to the substrate without the use of any chemical linkers and/or additional processes.

In some embodiments, the scaffold of the present invention contains undifferentiated and/or differentiated stem cells. A variety of stem cells of various types and stages of differentiation can be used in the invention and include but are not limited to, for example, totipotent, pluripotent, multipotent and unipotent stem cells. In some embodiments, the stem cell is an embryonic stem (ES) cell. In some embodiments, the stem cell is mammalian. In some embodiments, the stem cell is human. In some embodiments, the stem cell is a progenitor stem cell. In some embodiments, the stem cell is a mesenchymal stem cell. In some embodiments, the stem cell is a neural stem cell (NSC). In some embodiments, the stem cell is a hematopoietic stem cell, In some embodiments, the stem cell is a mammary stem cell, In some embodiments, the stem cell is an intestinal stem cell, In some embodiments, the stem cell is a endothelial stem cell, In some embodiments, the stem cell is a olfactory adult stem cell, In some embodiments, the stem cell is a neural crest stem cell, In some embodiments, the stem cell is a testicular stem cell,

Stem cells of various types and stages of differentiation can be incorporated into the scaffold of the present invention. Non-limiting examples of differentiated cells include osteocytes, chondrocytes, osteoblasts, fibroblast, keratinocytes, adipocytes, neurons, oligo-dendrocytes, astocytes, microglial cells, muscles cells, nerve cells and cardiac myocytes. In some embodiments, the differentiated cell is a bone cell. In some embodiments, the differentiated cell is a chondrocyte. In some embodiments, the differentiated cell is an osteocyte. In some embodiments, the differentiated cell is a nerve cell. In some embodiments, the differentiated cell is an osteoblast. In some embodiments, the differentiated cell is an adipocyte. In some embodiments, the differentiated cell is an oligodendrocyte cell.

In some embodiments, the stem cell is a mesenchymal stem cell (MSC), which can differentiate in vitro, into a variety of connective tissues or progenitor cells, including, but not limited to, mesodermal (osteoblasts, chondrocytes, tenocytes, myocytes and adipocytes), ectodermal (neurons, astrocytes) and endodermal (hepatocytes) derived lineages. MSCs encompass multipotent cells from sources other than marrow, including but not limited to, muscle, dental pulp, cartilage, synovium, synovial fluid, tendons, hepatic tissues, adipose tissue, umbilical cord, and blood, including cord blood. In some embodiments, the stem cell is human adipose-derived mesenchymal stem cells (hADMSCs).

Another aspect of the present invention provides a device comprising the above described scaffold. The device may be implantable, including for example a patch, matrix or tube. In an exemplary embodiment, the device is a patch-like scaffold, which is composed of graphene oxide disposed on nanofibers. Such a device may be cultured in vitro prior to implant so that the stem cells have differentiated into cells of interest. Alternatively, the device may also be implanted prior to the differentiation of the stem cells. Further, in some embodiment, the stem cells are partially differentiated into cells of interest before implant.

The scaffold or device of the present invention may also be part of a kit. Besides the scaffold, the kit may contain agents for treatment of disorders and injuries including for example, a drug for neural therapy, an anti-inflammatory agent, anti-apoptotic agent, or growth factor. The kits may further contain catheters, syringes or other delivering devices. The kits may further contain instructions containing administration protocols for the therapeutic regimens. The kit may also contain media formulations selected to promote differentiation to cells of interest (e.g. osteocytes or chondrocytes). Suitable media include, but are not limited to, adipogenic media, osteogenic media, chondrogenic media, myogenic media, neurogenic media, hepatogenic media.

### Methods of Stem Cell Differentiation

Another aspect of the present invention provides a method of directing stem cell differentiation comprising exposing scaffold described herein to a culture media for a period of time sufficient to allow the stem cells to differentiate into cells of interest. The term “directing differentiation of a stem cell” as used herein is taken to mean causing a stem cell to develop into one or more specific differentiated cell types. Suitable stem cells are as described above. In some embodiments, the stem cells are human adipose-derived mesenchymal stem cells (hADMSCs). In some embodiments, the stem cells are neural stem cells.

The invention applies to a variety of stem cells of various types and stages of differentiation, and cultured in media that promotes differentiation toward a particular type of cells. While stem cells exemplified herein are differentiated into neural cells, differentiation into any desired “cell of interest” is contemplated. Examples include, but are not limited to, osteocytes, chondrocytes, osteoblasts, fibroblasts, keratinocytes, adipocytes, tenocytes, myocytes, hepatocytes, neurons, oligodendrocytes, astocytes, microglial cells, muscles cells, nerve cells and cardiac myocytes. In some embodiments, the differentiated cell is a chondrocyte. In some embodiments, the differentiated cell is a bone cell. In some embodiments, the differentiated cell is a neural cell. In some embodiments, the differentiated cell is an osteocyte. In some embodiments, the differentiated cell is a cardiac myocytes. In some embodiments, the differentiated cell is a muscle cell. In some embodiments, the differentiated cell is a nerve cell. In some embodiments, the differentiated cell is an osteoblast. In some embodiments, the differentiated cell is an adipocyte. In some embodiments, the differentiated cell is a hepatocyte. In some embodiments, the differentiated cell is an ectodermal cell. In some embodiments, the differentiated cell is an oligodendrocyte.

The stem cells can be seeded before or after the scaffold is exposed to the culture medium. Prior to the contact with the scaffold, the stem cells may be in the culture medium or in a separate system such as a solution or a suspension.

In some embodiments, the stem cells are grown on a scaffold of the present invention in an appropriate culture medium under conditions that do not require implementation with growth factors or external stimulation, or combinations thereof. In some embodiments, the stem cells or progenitor cells on the composited are grown and differentiated in vitro.

The stem cells may be induced to differentiate to cells of interest by methods known in the art, for example by culturing in media without EGF and bFGF, and optionally with addition of soluble cues. The culture media can be any liquid or solid preparation made specifically for the growth, storage or transport of microorganisms or other types of cells. The variety of media that exist allow for the culturing of specific organisms and cell types, such as differential media, selective media, test media and defined media. Non-limiting examples of the variety of suitable culture media include chondrogenic, osteogenic, myogenic, neurogenic, adipogenic, and hepatogenic media.

In some embodiments, the culture media does not contain growth factors or external stimulation. Conventional methods of stem cell differentiation involve growth factors or external stimulants to achieve a synergistic effect since differentiation in medium generally occurs over prolonged periods of time. “Growth factors” include naturally occurring substances capable of stimulating cellular growth, proliferation and cellular differentiation. For example, bone morphogenetic protein-2 (BMP-2) is a growth factor that plays an important role in the differentiation of cells into bone and cartilage. Other non-limiting examples include leukemia inhibitory factor (LIF), epidermal growth factor (EGF), fibroblast growth factor (FGF), transforming growth factor-beta (TBF-β), insulin-like growth factor (IGF), and vascular endothelial growth factor (VEGF), human growth hormone, platelet-derived growth factor (PDGF), interleukins, cytokines or combinations thereof. “External factors” or “external stimulants” are external sources of mechanical, acoustic or electromagnetic energy that can stimulate cellular proliferation and differentiation. For example, radiowaves or electromagnetic radiation can be used to supply cells with the sufficient energy needed to promote cellular growth. The scaffold of the present invention, however, allows for efficient stem cell differentiation in the absence of growth factors and external stimulants.

In some embodiments, the culture media is a neural induction media which contains DMEM-F12, B27, at least one of Penicillin and Streptomycin, and optionally growth factors. In general, DMEM is a basal medium consisting of the typical Amino Acids, Glucose, pH indicator, Salts and Vitamins. DMEM:F12 is a 50:50 mixture of DMEM and Ham's F12 media that has proven to be useful in a wide range of cell culture applications, especially when supplemented with fetal bovine serum (FBS). B27 is modification of serum-free Neurobasal medium and is commercially available from Invitrogen. In exemplary embodiments, the percentage weight of B27 is about 1%, 2%, 3%, 4%, or 5%. Penicillin/Streptomycin may be in the amount of about 0.5%, 1%, 2%, or 3%. Exemplary growth factors include EFT, FGF and BDNF. The content of the culture media may be adjusted during the course of the differentiation process. For example, one or more new or existing component may be added to the media or reduced. Part or all of the media may be replaced.

In some embodiments, the stem cells selectively differentiate into oligo-dendrocytes. Various biomarkers can be utilized to examine the differentiation. For example, to confirm the selective differentiation of NSCs into oligodendrocytes as opposed to astrocytes or neurons, the upregulation in mRNA levels of the oligodendrocyte markers GalC and MBP can be detected, showing stronger levels than in neuronal markers TuJ1 and MAP2 and astrocyte marker GFAP.

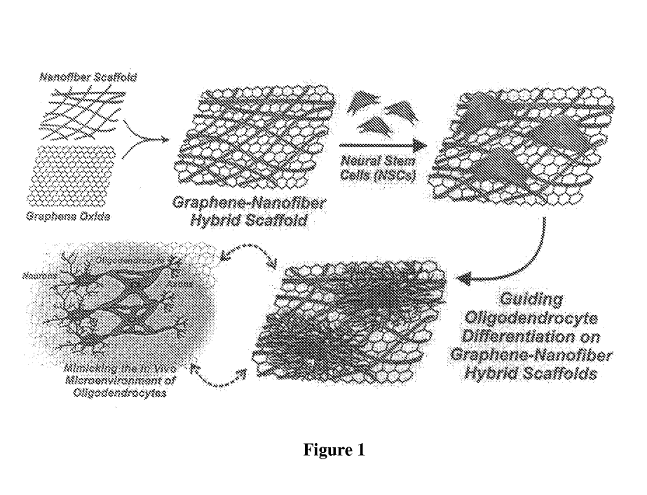

The use of GO as an effective coating material in combination with electrospun nanofibers for the selective differentiation of NSCs into oligodendrocytes can be illustrated in FIG. 1, which demonstrates the differentiation of NSC into oligodendrocytes under the guidance of a graphene oxide-based hybrid nanofibrous scaffold. Briefly, polymeric nanofibers (comprised of polycaprolactone) generated using electrospinning can be subsequently coated with graphene oxide (GO) and seeded with neural stem cells (NSCs). NSCs cultured on the graphene-nanofiber hybrid scaffolds show enhanced differentiation into oligodendrocyte lineage cells.

Another aspect of the invention provides a differentiated stem cell produced according to the method describe above. Also provided is an implantable medical device comprising the scaffold and the stem cells and/or differentiated stem cells, which are described in the method above.

### Methods of Dedifferentiation

The present invention also provides methods for inducing dedifferentiation of lineage committed mammalian cells into induced pluripotent stem cells (iPS cells). The method generally comprises: seeding a lineage committed mammalian cell in the scaffold of the present invention with or without growth factors; exposing the scaffold to a culture medium for a sufficient period of time to allow dedifferentiation of the cell. In some embodiments, the lineage committed mammalian cell is seeded in the scaffold prior to being exposed to the culture medium. In some embodiments, the lineage committed mammalian cell is seeded after the scaffold is exposed to the culture medium. In some embodiments, the scaffold is exposed simultaneously to the lineage committed mammalian cell and the medium. Before the contact with the scaffold, the lineage committed mammalian cell may be in the culture medium or in a separate system such as a solution or suspension. The induced pluripotent stem cells may originate from various types of lineage committed mammalian cells. Non-limiting examples of the lineage committed mammalian cells include osteocytes, chondrocytes, osteoblasts, fibroblasts, keratinocytes, adipocytes, tenocytes, myocytes, hepatocytes, neurons, oligodendrocytes, astocytes, microglial cells, muscles cells, nerve cells and cardiac myocytes. The lineage committed mammalian cells may be from an animal or human.

An important application of pluripotent cells is their use in cell therapy. Pluripotent stem cells include, but are not limited to, human embryonic stem (hES) cells, human embryonic germ (hEG) cells. Still other types of pluripotent cells exist, for example, dedifferentiated mouse and human stem cells, i.e. differentiated somatic adult cells are dedifferentiated to become pluripotent-like stem cells. These dedifferentiated cells (iPS cells) are induced to establish cells having pluripotency and growth ability similar to those of ES cells. Reprogramming of differentiated human somatic cells into a pluripotent state allows for patient- and disease-specific stem cells (see Han, et al., BioScience, 2010, 60 (4), 278-285; Takahashi, K. et al. Cell, 2007 1-12; and Ju, J. et al. Science 2007). Takahashi et al. and Ju et al. each introduced four genes into adult and fetal/newborn fibroblasts to generate the iPS cells: Oct4, Sox2, Klf4 and c-myc by Takahashi et al.; Oct4, Sox2, Nanog and Lin28 by Ju et al. In either case, iPS cells had some characteristics of hES cells including, hES cell morphology, marker expression, prolonged proliferation, normal karyotype, and pluripotency.

Various approaches in the literature can be adapted to the present invention for the production of iPS cells (Fan, et al., BioScience, 2010, 60 (4), 278). For example, certain nuclear reprogramming factors have allowed pluripotent stem cells or pluripotent-like stem cells to be derived from somatic cells. Nuclear programming factors are described in U.S. Patent Application Publication No. 2009/0047263, International Patent Application Publication No. WO2005/80598, U.S. Patent Application Publication No. 2008/0233610 and International Patent Application Publication No. WO2008/11882 and were used to induce reprogramming of a differentiated cell without using eggs, embryos, or ES cells. Methods for preparing induced iPS cells from somatic cells by using the nuclear reprogramming factor similar to that used and described in the present invention are not particularly limited. In some embodiments, the nuclear reprogramming factor contacts the lineage committed mammalian cells under an environment in which the somatic cells and induced pluripotent stem cells can proliferate. An induced pluripotent stem cell can be prepared by contacting a nuclear reprogramming factor with a lineage committed mammalian cell in the absence of eggs, embryos, or embryonic stem (ES) cells. By using a nuclear reprogramming factor, the nucleus of a lineage committed mammalian cell can be reprogrammed to obtain an iPS cell or an “ES-like cell.”

Many of the known methodologies employ in the culture medium certain reprogramming factors comprising expression cassettes encoding Sox-2, Oct-4, Nanog and optionally Lin-28, or expression cassettes encoding Sox-2, Oct-4, Klf4 and optionally c-myc, or expression cassettes encoding Sox-2, Oct-4, and optionally Esrrb. Nucleic acids encoding these reprogramming factors can be in the same expression cassette, different expression cassettes, the same reprogramming vector, or different reprogramming vectors. For example, Oct-3/4 and certain members of the Sox gene family (Sox-1, Sox-2, Sox-3, and Sox-15) are crucial transcriptional regulators involved in the induction process. Oct-3/4 (Pou5f1) is one of the family of octamer (“Oct”) transcription factors, and plays an important role in maintaining pluripotency.

Various agents can be used to mimic the effects of the transcription factors and induce the dedifferentiation. For example, small molecules have been applied to inducing dedifferentiation (see for example, Chen, et al., J. Am. Chem Soc., 2004, 126, 410-411; Huangfu, et al., Nat Biotechnol, 2008, 26 (7), 795-7; Hou, et al., Science, 2013, 341 (6146), 651-654). Histone deacetylase (HDAC) inhibitors have been reported to enhance the production of iPS cells (Huangfu, et al., Nat. Biotechnol. 2008, 26, 1269). Other exemplary small molecules include Pluripotin, BIO (GSK 3β inhibitor), ID 8, TWS 119, Reversine, Trichostain A, Vaproic Acid, 5-Azacytidine, BIX 01294, Bay K 8644, and A83-01 (see for example Christie, et al., Tocris Reviews No. 37, Tocris Bioscience) The condition of the differentiation process depends on factors such as the specific lineage committed mammalian cell and the specific type of scaffold. In exemplary embodiments, the scaffold is exposed to a small molecule for induction of dedifferentiation process, before, after, or simultaneously with the seeding of the lineage committed mammalian cells into the scaffold. The small molecule may also be introduced to the scaffold before, after, or simultaneous with the exposure of the scaffold to the culture medium. As described above, the culture medium may or may not contain growth factors and external stimulation. One or more small molecules of different structures may be used in combination. One of ordinary skill in the art is able to identify the optimal condition in view of the knowledge available in the art without undue experiments.

Dedifferentiation may also be induced in hypoxic conditions. The media components are generally dictated by the growth requirements of the lineage committed mammalian cells used as the starting cells (see for example WO2009142717). Other suitable conditions/factors have been reported in the literature and can be readily applied to the present invention (see for example Yang, et al., Eur J Pharmacol. 2014, 734, 83-90).

Another aspect of the present invention provides iPS cells produced according to the above described method (Perkel, Science, 2015, 347 (6227), 1271; Sun, et al., Sci. Transl. Med. 2012, 4 (130), 130). Also provided is a method of treating a disease or disorder comprising administering the iPS cells or implanting the scaffold containing the iPS cells, wherein the iPS cells are produced according to the above described method. The method is applicable to the above described diseases and disorders and various other patient specific diseases. The terms “administration of” and or “administering ” should be understood to mean providing a cell of the invention or a mixture comprising the cell of the invention to the subject in need of treatment. “Administering” includes, but is not limited to, subcutaneous insertion, topical application, intradermal injection, intravenous injection and subcutaneous injection.

Another aspect of the invention provides a iPS cell produced according to the method describe above. Also provided is an implantable medical device comprising the scaffold and iPS cells, which are described in the method above.

### Method of Treating an Injury or Disorder

Another aspect of the present invention provides a method of treating or ameliorating a disorder by implanting the above described scaffold to a subject in need. The scaffold may contain one or more types of stem cells or differentiated stem cells (cells of interest differentiated from the stem cells). The subject is a mammal and can be an animal or human. In some embodiments, the scaffold is implanted in a subject in need to stimulate growth and/or repair of bone, cartilage, muscle, or nervous tissue in a host. Prior to implantation, the scaffold may contain stem cells or differentiated stem cells (e.g. osteoblasts or neural cells).

The selective stem differentiation process of the present invention allows for tissue engineering or regeneration in the treatment of various diseases or disorders, which also include wounds caused by disease, trauma, surgery, burns and bites. Exemplary tissue engineering include cardiac muscle regeneration, neural tissue regeneration, vascular regeneration, and bone tissue regeneration. The term “implanting” in tissue engineering includes, but is not limited to, subcutaneous insertion, topical application, intradermal injection, intravenous injection and subcutaneous injection.

In some embodiments, the present invention provides methods of treating or ameliorating a neurodegenerative disorder or a neurological injury comprising implanting a scaffold of the present invention to a subject in need of such treatment. Neurodegenerative disorders and neurological injuries include for example conditions of neuronal cell death or compromise, and include acute and chronic disorders of the central and peripheral nervous system. Such disorders and injuries include, without limitation, traumatic brain injury, spinal cord injury, peripheral nerve trauma, Alzheimer's Disease, Parkinson's Disease, Huntington's Disease, epilepsy, stroke and dementias. The scaffold can be delivered to a site in the central or peripheral nervous system in proximity to an area of damaged neural tissue by methods known in the art, for example by implantation. The scaffold may be delivered simultaneously with, before, or after another agent including for example, a drug for neural therapy, an anti-inflammatory agent, anti-apoptotic agent, or growth factor.

In some embodiments, the present invention provides methods of treating or ameliorating a bone-related disorder or injury by implanting a scaffold of the present invention to the site of injury or disorder in a mammal. A bone disorder may be any disorder characterized by a net bone loss (osteopenia or osteolysis).

Non-limiting examples of bone disorder include: Osteoporosis, such as primary osteoporosis, endocrine osteoporosis (hyperthyroidism, hyperparathryoidism, Cushing's syndrome, and acromegaly), hereditary and congenital forms of osteoporosis (osteogenesis imperfecta, homocystinuria, Menkes' syndrome, and Riley-Day syndrome) and osteoporosis due to immobilization of extremities; Paget's disease of bone (osteitis deformans) in adults and juveniles; Osteomyelitis, or an infectious lesion in bone, leading to bone loss; hypercalcemia resulting from solid tumors (breast, lung and kidney) and hematologic malignacies (multiple myeloma, lymphoma and leukemia), idiopathic hypercalcemia, and hypercalcemia associated with hyperthryoidism and renal function disorders; Osteopenia following surgery, induced by steroid administration, and associated with disorders of the small and large intestine and with chronic hepatic and renal diseases; Osteonecrosis, or bone cell death, associated with traumatic injury or nontraumatic necrosis associated with Gaucher's disease, sickle cell anemia, systemic lupus erythematosus and other conditions; bone loss due to rheumatoid arthritis; Periodontal bone loss; Osteolytic metastasis.

### Method of Coating Nanofibers

Another aspect of the present invention provides a method for preparing the above described scaffolds. The method includes contacting the nanomaterial such as nanofibers with graphene oxide. The graphene oxide may be prepared as a solution, which is then disposed on the nanomaterial. In some embodiments, the solution is sprayed or brushed onto the nanofibers. In some embodiments, the nanofibers are immersed into a solution of graphene oxide for a desirable period of time. The exact condition for coating the nanofibers can be determined by one of ordinary skill in the art without undue experiments. Also provided is a scaffold prepared according to the above described method.

In some embodiments, the coating on the nanofibers is adjusted by controlling for example the concentration of the graphene oxide solution, the length in time for the coating, and the temperature of the coating solution and/or nanofibers. In some embodiments, the graphene oxide coating on the nanofibers reaches a saturation point or a maximum amount.

### EXAMPLES

### Electro Spinning PCL Nanofibers

Polycaprolactone (PCL, 80 kDa, Sigma, cat. #440744) was dissolved in a 3:1 (v/v) mixture of chloroform-methanol to prepare a 5% (w/v) polymer solution. The solution was placed into a syringe with a 22-gauge needle and electrospun onto an aluminum surface, which was positioned horizontally, at a flow rate of 0.8 mL/hr. A 20-kV voltage was applied with a high voltage power supply and a 15-cm working distance was utilized. The scaffolds were then dried under vacuum for two days, and then transferred to cover glass (No. 1, VWR) using a medical grade adhesive (Factor 2, cat. #B400).

### Synthesis of Graphene Oxide

Thin-layered GO was synthesized by first making graphite oxide using a modified Hummer's method. Briefly, graphite (1 g; Sigma Aldrich, 100 mesh) was mixed with 98% H₂SO₄ (12 mL), K₂S₂O₈ (2.5 g) and P₂O₅ (2.5 g) at 80° C. on a hotplate for six hrs. Then, de-ionized water (500 mL) was added into the mixture and the solution was stirred overnight. The preoxidized graphite was filtered out by using filter paper with 200-nm pores. After dried under ambient conditions overnight, graphite with pre-treatment was stirred with concentrated H₂SO₄ (98%). After 10 mins, KMnO₄ (15 g) was slowly added into the mixture in a 30 min time period with stirring under the ice bath condition (temperature was kept below 15° C.). After the addition of KMnO₄, the temperature of the mixture was gradually raised to 35° C. and was stirred for three hours. Successively, de-ionized water (250 ml) was slowly dropped into the mixture (temperature below 50° C.) with vigorous stirring, followed by stirring for four hours. Finally, the reaction was quenched by the addition of de-ionized water (700-ml) followed by the addition of 30% H₂O₂ (20 ml). The graphite oxide was centrifuged down under 10000 rpm for 10 minutes and washed with 10% HCl solution (three times) and de-ionized water (five times) to get the graphite oxide. A two hour ultrasonication process (40 kHz, 40 W, 1 second ultrasonication and 1 second resting period) was used to exfoliate the graphite oxide aqueous solution into graphene oxide (GO). Finally, the GO solution was centrifuged under 13300 rpm for 30 minutes to get rid of multi-layered GO.

### Generating GO-Coated PCL Hybrid Scaffolds

GO was dispersed in deionized water at varying concentrations (0.1, 0.5 and 1.0 mg/mL). The substrates (cover glass or PCL nanofibers) were treated with oxygen plasma for one min, followed by deposition of the GO solution directly on top of the substrate for five minutes. Substrates were then vacuum-dried for at least six hr. The Renishaw inVia Raman microscope was used to quantify the amount of GO-coating. After gold sputtering, the Zeiss Sigma field emission scanning electron microscope (FE-SEM) and the ORION™ helium ion microscope was used to visualize the scaffolds.

### Rat NSC Culture & Differentiation

GFP-labeled rat neural stem cells (Millipore) were purchased and expanded according to the manufacture's protocol. The NSCs were maintained in laminin (Sigma, 10 μg/ml) coated culture dishes precoated with poly-L-lysine (PLL, 10 μg/ml) in Millitrace basal media (Millipore) supplemented with the penicillin and streptomycin (Life Technologies), in the presence of basic fibroblast growth factor (bFGF-2, 20 ng/ml, Millipore). All of the cells were maintained at 37° C. in a humidified atmosphere of 5% CO2. For consistency, the experiments were carried out on cells between passages 2 and 5. In preparation for NSC culture, the substrates were treated with oxygen plasma for 1 min and then coated with laminin (10 μg/mL) overnight in the culture hood. While oxygen plasma treatment was observed to be sufficient for sterilization, substrates were alternatively sterilized under UV for 30 minutes prior to laminin coating. Control glass substrates were coated with PLL (10 μg/ml) overnight followed by laminin (10 μg/mL) overnight. NSCs were cultured on the substrates at 0.8×105 NSCs/mL in basal medium (without bFGF) to initiate differentiation. The cells were allowed to differentiate for six days, with the basal medium exchanged every other day. After six days of culture, a significant difference in the cellular morphology was evident on GO-coated nanofibers compared to the nanofibers alone. FE-SEM shows cell attachment on these surfaces in greater detail, wherein the cells on the GO-coated nanofibers display extensive branching of cell processes. This type of extensive process extension is a characteristic attribute reported to distinguish oligodendrocytes from other neural cells. This difference in cellular morphology provides evidence for the potential ability of our hybrid scaffolds to enhance NSC differentiation into oligodendrocytes.

### Stem Cell Differentiation on GO-Coated PCL

To systematically investigate the effect of GO-coating on NSC differentiation, hybrid scaffolds with varying amounts of GO-coating were generated. Solutions containing three different concentrations of GO (0.1, 0.5 and 1.0 mg/mL) were deposited on oxygen plasma-treated PCL nanofibers. The degree of coating using the various GO concentrations was then observed using FE-SEM. GO-coating of PCL with 0.1 mg/mL, indicated as PCL-GO (0.1), shows the clear presence of GO compared to PCL nanofibers alone, with uniform coating on the surface of individual fibers. In contrast, PCL-GO (0.5) and PCL-GO (1.0) exhibit a much greater extent of GO attachment on the nanofibrous surface, showing a degree of GO coating and connectivity between fibers. This was confirmed quantitatively using Raman Spectroscopy, where the characteristic peaks of the D band (˜1350 cm−1) and G band (˜1600 cm−1) indicate the presence of GO. Comparison of the Raman intensity of these peaks further supports the trend described above in terms of concentration-dependent GO coating on the PCL nanofiber surfaces. Moreover, the nanofibrous scaffolds at all three concentrations show significantly higher GO content compared to control glass surfaces coated with the same respective amounts of GO. The higher surface area-to-volume of the nanofibers available for GO attachment, in conjunction with the 3D structure of these scaffolds, may attribute to this difference in coating.

These various PCL-GO substrates were then used to examine the influence of GO-coating on modulating NSC differentiation. For comparison, the following control substrates were used: 1) PLL-coated glass (conventional substrate for in vitro neural cultures), 2) PCL nanofibers alone, and 3) GO-coated glass (at the abovementioned three GO concentrations). All of the substrates were coated with laminin to facilitate NSC attachment, and the cells were harvested after six days of culture to compare the gene expression of key neural markers. Quantitative PCR (qPCR) was utilized to compare gene expression of three key markers that are indicative of differentiated NSCs: glial fibrillary acidic protein (GFAP; astrocytes), beta-III tubulin (TuJ1; neurons) and myelin basic protein (MBP; mature oligodendrocytes). First, it is important to note that both the PCL nanofibers alone and GO-coated glass (at all three concentrations) individually show enhanced oligodendrocyte gene expression, with about a 2-fold increase in MBP expression. At the same time, TuJ1 shows only about a 1.3-fold increase and GFAP shows about a 0.5-fold decrease in expression, which indicates a stronger preference for differentiation towards oligodendrocytes rather than neurons and astrocytes.

The synergistic effect resulting from the combination of GO and nanofibers in a single scaffold was studied. In the PCL-GO samples, a remarkable trend in gene expression of these neural markers was observed. The nanofibers coated at the lowest GO concentration (0.1 mg/mL) showed a 6.5-fold increase in MBP, which is much higher than the expression on PCL nanofibers alone and GO-coated glass controls. Interestingly, this enhancement in MBP expression was even more pronounced when the concentration of GO was further increased, wherein the cells on PCL-GO (0.5) showed an 8.9-fold increase and PCL-GO (1.0) showed a 9.9-fold increase in MBP expression. Based on the data, there is no statistically significant difference in MBP expression on the PCL-GO (0.5) and PCL-GO (1.0), indicating the saturation of GO on the PCL nanofiber surface. The overall increase in MBP expression of the cells grown on the PCL-GO substrates points to the role of GO in the observed result, in which the 3D PCL nanotopography serves to increase the amount of GO coating and the consequent surface interface in contact with the NSCs compared to the traditional 2D surfaces. In addition, the simultaneous decrease in GFAP expression and relatively small increase in TuJ1 expression provides further evidence that the hybrid scaffold promotes selective NSC differentiation, with a strong preference towards oligodendrocyte lineage cells. To explore the potential of these hybrid scaffolds as a culture platform for oligodendrocyte differentiation, we elected to use PCL-GO (1.0) for all subsequent experiments (termed PCL-GO hereafter). In regard to biocompatibility, NSCs grown on these scaffolds show excellent survival, as found with cell viability assays.

We next sought to further characterize the degree of differentiation into oligodendrocytes by examining the expression of well-established oligodendrocyte markers at the genetic- and cellular-level. After six days of culture, the cells grown on PCL-GO were immunostained for the early marker Olig2 and the mature marker MBP. The immunostained cells show extensive expression of both the nuclear-localized Olig2 and the cytosolic MBP. A similar expression was also observed for the oligodendrocyte-specific surface markers O4 (early) and GalC (mature). Expression of these protein markers confirms the successful NSC differentiation into oligodendrocytes. The degree of differentiation was further quantified by determining the percentage of cells expressing Olig2 and MBP on the various substrates. While the conventional PLL-coated glass substrates showed only about 9% of the cells expressing Olig2, both the PCL only and GO-coated glass substrates showed about 16% Olig2-expressing cells. On the other hand, the PCL-GO substrate displayed about 33% of the cells expressing Olig2, which is significantly higher than all other conditions. A similar trend was also observed for MBP expression, wherein 26% of the cells on PCL-GO were positive for MBP, which corroborates the gene expression results shown earlier. Comparison of the percentage of cells stained for TuJ1 (neurons) and GFAP (astrocytes) further supports the selective differentiation into oligodendrocytes, with PCL-GO displaying a significant decrease in GFAP-positive cells and a minor increase in the number of TuJ1-positive cells. Given the difficulty in achieving the spontaneous differentiation of stem cells into oligodendrocytes, our unique graphene-nanofiber hybrid scaffolds exhibit a significant enhancement in oligodendrocyte formation.

To further confirm that the hybrid scaffolds promote oligodendrocyte differentiation, we evaluated changes in gene expression for a variety of well-known early and mature oligodendrocyte-specific markers. qPCR was carried out for detecting the gene expression of: 1) early markers including 2′,3′-cyclic-nucleotide 3′-phosphodiesterase (CNP), platelet-derived growth factor receptor alpha (PDGFRα), Olig1 and Olig2, and 2) mature markers including proteolipid protein (PLP), MBP, myelin-associated glycoprotein (MAG), myelin oligodendrocyte glycoprotein (MOG), adenomatous polyposis coli (APC), glutathione S-transferase-pi (GST-π) and galactocerebroside (GalC). For all genes of interest, NSCs on PCL-GO exhibited the strongest level of expression compared with all other control substrates. Interestingly, several of the known genes indicative of myelinating oligodendrocytes also showed a substantial increase in gene expression. For instance, MAG and MOG, which are glycoproteins reported to be crucial during the myelination process in the CNS, were seen to have a 17-fold and 19-fold increase in gene expression, respectively. Taken together, these results confirm that NSCs cultured on PCL-GO substrates exhibit a strong preference towards oligodendrocyte differentiation.

### Promotion of Oligodendrocyte Differentiation-Related Signal Transduction

Among the various cell signaling proteins, we examined the expression of FAK, Akt, ILK and Fyn, which have been found to mediate cytoskeletal remodeling and process extension during oligodendrocyte development. Moreover, disruption of each of these proteins has been reported to cause a variety of developmental defects including reduced process extension, aberrant myelin formation and attenuated expression of myelin proteins. We found that NSCs cultured on the GO-coated surfaces enhanced the gene expression of all of these factors. These signaling molecules exhibited the same trend in expression, wherein the GO-coated glass showed higher expression than PCL, and PCL-GO showed the strongest level of expression with a 2.6-fold increase in FAK and about a 1.7-fold increase in Akt, ILK and Fyn. Additionally, treating the cells grown on PCL-GO scaffolds with cell signaling inhibitors showed a significant decrease in gene expression of mature oligodendrocyte markers, which provides further evidence for the potential role of such cellular signaling in the observed oligodendrocyte differentiation. Collectively, this data supports the role of GO-coating in the upregulation of these downstream molecules in the integrin signaling pathway and may explain, at least in part, the enhanced oligodendrocyte differentiation of NSCs on our hybrid scaffolds.

In order to further elucidate this correlation, we sought to observe cellular co-localization of markers indicative of both integrin signaling and oligodendrocyte differentiation using confocal microscopy. Dual staining was carried out for: 1) Olig2, an oligodendrocyte marker, and 2) FAK, one of the main regulators of integrin-ECM signaling and found in our study to show the highest expression in cells cultured on PCL-GO. The immunostaining for Olig2 (purple) and FAK (orange) was compared for NSCs cultured on PCL-GO with the other control substrates. As observed earlier, cells grown on PCL-GO showed the strongest intensity and highest number of cells expressing Olig2, with minimal expression on the glass control and moderate expression on PCL and GO. A similar trend was also observed in FAK staining. Since the localization of FAK is in the cytoplasm and Olig2 is in the nucleus, the co-localization of the two markers within the same cell can be easily visualized. Interestingly, the cells expressing FAK also expressed Olig2, a phenomenon that was observed on all substrates. Moreover, PCL-GO showed the strongest expression of both markers and the highest number of cells co-expressing FAK and Olig2. Together, our data suggests that the GO-coating on the nanofiber scaffolds may promote oligodendrocyte differentiation through specific microenvironmental interactions which activate integrin-related intracellular signaling.

### Immunocytochemistry

Cell cultures were fixed with 4% formaldehyde (ThermoScientific) for 15 min, blocked for 1 hr with 5% normal goat serum (NGS, Life Technologies), and permeabilized with 0.3% Triton X-100 when staining for intracellular markers (MBP, Olig2, Tun, GFAP & FAK). The combinations of the following primary antibodies were incubated overnight at 4° C.: mouse antibody to Olig2 (1:300, Millipore, cat. #MABN50), mouse antibody to MBP (1:300, AbCam, cat. #ab62631), mouse antibody to O4 (1:50, StemCell Technologies, cat. #01416), mouse antibody to GalC (1:200, Millipore, cat. #MAB342), mouse antibody to TuJ1 (1:500, Covance, cat. #MMS-435P), rabbit antibody to GFAP (1:300, Dako, cat. #Z033429-2) and rabbit antibody to FAK (1:75, Santa Cruz Biotech, cat. #sc-557). Appropriate Alexa Fluor 546- and Alexa Fluor 647-conjugated secondary antibodies were used to detect the primary antibodies (1:200, Molecular Probes) and Hoechst 33342 (1:100, Life Technologies) as a nuclear counterstain. The substrates were mounted on glass slides using ProLong® Gold antifade (Life Technologies) and imaged using either the Nikon TE2000 Fluorescence Microscope or Zeiss LSM 710 Confocal Microscope.

### Cell Viability

Cell viability of the cells on the various substrates (PLL-coated glass, GO-coated glass, PCL and PCL-GO) was determined after six days of culture using MTS Assay (Promega). All experiments were conducted in triplicates and the percentage of viable cells was determined following standard protocols described by the manufacturer. The data is represented as formazan absorbance at 490 nm, and normalized to the conventional PLL-coated glass control.

### PCR Analysis

Total RNA was extracted using Trizol Reagent (Life Technologies) and the mRNA expression levels were analyzed using quantitative PCR (qPCR). Specifically, cDNA was generated from 1 μg of total RNA using the Superscript III First-Strand Synthesis System (Life Technologies). Analysis of mRNA was then accomplished using primers specific to each of the target mRNAs. qPCR reactions were performed using SYBR Green PCR Master Mix (Applied Biosystems) in a StepOnePlus Real-Time PCR System (Applied Biosystems) and the resulting Ct values were normalized to GAPDH. Standard cycling conditions were used for all reactions with a melting temperature of 60° C. The primer sequence for the genes which were analyzed is provided below:

| | | | Size |
|---|---|---|---|
| Gene | F Primer | R Primer | (bp) |
| GAPDH | ATGACTCTACCCACGGCAAG | GGAAGATGGTGATGGGTTTC | 87 |
| TUJ1 | ACTTTATCTTCGGTCAGAGTG | CTCACGACATCCAGGACTGA | 97 |
| GFAP | GAGAGAGATTCGCACTCAGTA | TGAGGTCTGCAAACTTGGAC | 89 |
| GALC | GAAGGTCTCCAGCGAGTGAG | TCAAGCAGCACAGAAGAGGA | 74 |
| MBP | CACAAGAACTACCCACTACGG | GGGTGTACGAGGTGTCACAA | 103 |
| CNP | AGGGGCTTATCTCTCACCGT | AACCAGAGATGTGGCTTCCG | 117 |
| PDGFRα | TGGAAATGGACGGACAAGGG | TGGGAATCTCACCAATGCCC | 76 |
| OLIG1 | GTTAACCACAGCAAGGCAGC | TCGGCTACTGTCAACAACCC | 178 |
| OLIG2 | GAACCCCGAAAGGTGTGGAT | TTCGATTTGAGGTGCTCGCT | 93 |
| PLP | GCCACACTAGTTTCCCTGCT | ATCAGAACTTGGTGCCTCGG | 91 |
| MAG | CACACAAGTGGTCCACGAGA | GCTCCGAGAAGGTGTACTGG | 164 |
| MOG | TGTGTGGAGCCTTTCTCTGC | CCCAGGAGATATACGGCACG | 160 |
| APC | TACTTCATCGTCCACGCAGC | ACAATGGTGTACGGTGGCAT | 72 |
| GST-π | GTCCACACCTCTGTCTACGC | GGACTTGAGCGAGCCTTGAA | 165 |
| FAK | CAATGCCTCCAAATTGTCCT | TCCATCCTCATCCGTTCTTC | 157 |
| AKT | GCCACGGATACCATGAACGA | AGCTGACATTGTGCCACTGA | 197 |
| ILK | GGGCTCTTGTGAGCATCTGT | TGTTCAGGGTCCCATTTCGG | 183 |
| FYN | GGTGGGGAACGGACTCATTT | CCAAAGGACCACACGTCAGA | 168 |

It will be appreciated by persons skilled in the art that the present invention is not limited to what has been particularly shown and described. Rather, the scope of the present invention is defined by the claims which follow. It should further be understood that the above description is only representative of illustrative examples of embodiments. The description has not attempted to exhaustively enumerate all possible variations. The alternate embodiments may not have been presented for a specific portion of the invention, and may result from a different combination of described portions, or that other un-described alternate embodiments may be available for a portion, is not to be considered a disclaimer of those alternate embodiments. It will be appreciated that many of those un-described embodiments are within the literal scope of the following claims, and others are equivalent.

1. A scaffold for tissue engineering comprising nanofibers coated with graphene oxide, wherein said nanofibers have an average diameter in the range of about 100 nm-3 μm.

2. The scaffold of claim 1, wherein the nanofibers comprise a polymer selected from the group consisting of polyglycolic acid (PGA), polylactic acid (PLA), poly(lactic-co-glycolic acid) (PLGA), poly-ε-caprolactone (PCL), polyanhydride, polyorthoesters, polyvinylalcohol, polyethylene glycol, polyurethane, polyacrylic acid, poly-N-isopropyl acrylamide, poly(ethylene oxide)-poly(propylene oxide)-poly(ethylene oxide)copolymers, derivatives thereof, and copolymers thereof.

3. The scaffold claim 2, wherein the polymer is PCL.

4. The scaffold of claim 1, wherein the lateral dimension of the graphene oxide is in the range of about 50-1000 nm.

5. The scaffold of claim 1, wherein the graphene oxide is saturated on the nanofibers.

6. The scaffold of claim 1, further comprising stem cells seeded in the scaffold.

7. The scaffold of claim 6, wherein the stem cells are selected from the group consisting of pluripotent stem cells (PSCs), mesenchymal stem cells (MSCs) and neural stem cells (NSCs).

8. The scaffold of claim 1, further comprising differentiated stem cells seeded in the scaffold.

9. The scaffold of claim 8, wherein the differentiated stem cells comprise at least one member selected from the group consisting of osteocytes, chondrocytes, osteoblasts, fibroblasts, keratinocytes, adipocytes, tenocytes, myocytes, hepatocytes, neurons, oligodendrocytes, astocytes, microglial cells, muscles cells, nerve cells and cardiac myocytes.

10. An implantable medical device comprising the scaffold of claim 1.

11. A method of directing stem cell differentiation comprising exposing the scaffold of claim 1 to a culture media comprising stem cells for a period of time sufficient to allow the stem cells to differentiate into cells of interest.

12. The method of claim 10, wherein the stem cells are selected from the group consisting of pluripotent stem cells (PSCs), mesenchymal stem cells (MSCs) and neural stem cells (NSCs).

13. The method of claim 10, wherein the stem cells differentiate into at least one member selected from the group consisting of osteocytes, chondrocytes, osteoblasts, fibroblasts, keratinocytes, adipocytes, tenocytes, myocytes, hepatocytes, neurons, oligodendrocytes, astocytes, microglial cells, muscles cells, nerve cells and cardiac myocytes.

14. The method of claim 10, wherein the culture media does not contain growth factors or external stimulation.

15. A method of treating an injury or disorder comprising implanting the scaffold of claim 1 in a subject in need, wherein the scaffold is seeded with stem cells or differentiated stem cells.

16. The method of claim 15, wherein the subject is human.

17. The method of claim 15, wherein the stem cells are selected from the group consisting of pluripotent stem cells (PSCs), mesenchymal stem cells (MSCs) and neural stem cells (NSCs).

18. The method of claim 15, wherein the differentiated stem cells comprise at least one member selected from the group consisting of osteocytes, chondrocytes, osteoblasts, fibroblasts, keratinocytes, adipocytes, tenocytes, myocytes, hepatocytes, neurons, oligodendrocytes, astocytes, microglial cells, muscles cells, nerve cells and cardiac myocytes.

19. A method of preparing a scaffold of claim 1, comprising contacting nanofibers with a solution of graphene oxide, wherein the concentration of the graphene oxide is optionally adjusted to control the thickness of the coating.

20. The method of claim 10, wherein the graphene oxide deposited on the nanofibers reaches saturation.

21. A method of dedifferentiating lineage committed mammalian cells into induced pluripotent stem cells (iPS cells), comprising seeding lineage committed mammalian cells in the scaffold of claim 1 and exposing the scaffold to a culture medium for a sufficient period of time to allow dedifferentiation of the lineage committed mammalian cells.

22. The method of claim 21, wherein the lineage committed mammalian cells comprise at least one member selected from the group consisting of osteocytes, chondrocytes, osteoblasts, fibroblasts, keratinocytes, adipocytes, tenocytes, myocytes, hepatocytes, neurons, oligodendrocytes, astocytes, microglial cells, muscles cells, nerve cells and cardiac myocytes.

23. An iPS cell produced according to the method of claim 21.

24. A method of treating a disease or disorder comprising administering iPS cells produced according to the method of claim 21 to a subject in need.
